(19) United States
(12) Patent Application Publication
(10) Pub. No.: US 2022/0356137 A1
(43) Pub. Date: Nov. 10, 2022

(54) METHOD OF ENHANCED AROMATIC SELECTIVITY FOR GAS PHASE DEOXYGENATION OF BIO-OILS

(71) Applicants: University of Kansas, Lawrence, KS (US); Purdue Research Foundation, West Lafayette, IN (US)

(72) Inventors: Bala Subramaniam, Lawrence, KS (US); Anand Ramanathan, Bartlesville, OK (US); Yang Xiao, West Lafayette, IN (US); Arvind Varma, West Lafayette, IN (US)

(21) Appl. No.: 17/621,259

(22) PCT Filed: Jun. 19, 2020
(86) PCT No.: PCT/US20/38637

§ 371 Date: Dec. 21, 2021

Related U.S. Application Data
(60) Provisional application No. 62/865,209, filed on Jun. 22, 2019.

(51) Int. Cl. C07C 37/055; B01J 23/648; B01J 23/652; B01J 29/03

(52) U.S. Cl. CPC C07C 37/055; B01J 23/6484; B01J 23/6527; B01J 29/0308

(57) ABSTRACT

Methods for gas-phase deoxygenation of a bio-oil are provided. In embodiments, such a method comprises exposing a bio-oil vapor comprising hydrocarbon compounds having oxygenated aromatic groups, to hydrogen gas in the presence of catalyst under conditions to induce deoxygenation of the oxygenated aromatic groups to provide a deoxygenated aromatic species, wherein the catalyst is a transition metal-incorporated mesoporous silicate having platinum deposited thereon and the transition metal is selected from Nb, W, Zr, and combinations thereof. The transition metal-incorporated mesoporous silicate catalysts are also provided.

### CROSS-REFERENCE TO RELATED APPLICATIONS

The present application claims priority to U.S. provisional patent application No. 62/865,209 that was filed Jun. 22, 2019, the entire contents of which are incorporated herein by reference.

### REFERENCE TO GOVERNMENT RIGHTS

This invention was made with government support under 1539105 awarded by the National Science Foundation. The government has certain rights in the invention.

### BACKGROUND

Pyrolysis-derived biofuels from biomass sources are called bio-oils, which, as compared to petroleum-based fossil fuels, contain much higher oxygen content, thus cannot be used directly. Catalytic hydrodeoxygenation (HDO) offers a plausible route to obtain upgraded bio-oils. Owing to the complex composition (hundreds of species) of bio-oils, model compounds are often used to investigate bio-oil upgrading for catalyst development purposes. In this context, guaiacol, containing both hydroxyl (—OH) and methoxy (—OCH₃) groups, has been extensively employed as a model compound. Various catalytic materials have been reported for the HDO of guaiacol, including supported transition metals such as Pt, Ru, Co, and Ni. However, these catalysts typically form partially deoxygenated species such as phenol and anisole (methoxybenzene) under 0.1 MPa hydrogen pressure and temperatures ranging from 250-500 ° C. Fully deoxygenated species such as toluene and cyclohexane require elevated hydrogen pressures (e.g. 3-5 MPa) and temperatures 200-300 ° C.

Bifunctional catalysts, containing both metallic sites and acidic sites in a single catalyst, have also shown guaiacol HDO activity to deoxygenated species. Such studies typically employed noble metals supported over microporous zeolites for liquid-phase guaiacol HDO under elevated H₂ pressure (e.g. 3-5 MPa) and relatively low temperature (<250° C.). Several issues of these bifunctional catalysts, however, have been identified, including relatively low conversion of guaiacol, requirement of high operating H₂ pressure for full hydrodeoxygenation, and challenges for catalyst stability caused by coking/carbon deposit. In addition, the mechanistic steps in the overall hydrodeoxygenation reaction and the catalytically active sites (metallic vs acidic) responsible for guaiacol full HDO over these bifunctional catalysts are not well understood.

### SUMMARY

Provided are methods for gas-phase deoxygenation of a bio-oil. In embodiments, such a method comprises exposing a bio-oil vapor comprising hydrocarbon compounds having oxygenated aromatic groups, to hydrogen gas in the presence of catalyst under conditions to induce deoxygenation of the oxygenated aromatic groups to provide a deoxygenated aromatic species, wherein the catalyst is a transition metal-incorporated mesoporous silicate having platinum deposited thereon and the transition metal is selected from Nb, W, Zr, and combinations thereof.

The catalysts for use in the methods are also provided. In embodiments, such a catalyst is a transition metal-incorporated mesoporous silicate having platinum deposited thereon and the transition metal is selected from Nb, W, Zr, and combinations thereof.

Other principal features and advantages of the disclosure will become apparent to those skilled in the art upon review of the following drawings, the detailed description, and the appended claims.

### BRIEF DESCRIPTION OF THE DRAWINGS

Illustrative embodiments of the disclosure will hereafter be described with reference to the accompanying drawings.

FIGS. 1A-1F show initial performance of guaiacol hydrodeoxygenation at 300° C. and atmospheric pressure (0.1 MPa), H₂: guaiacol molar ratio=10 over (FIG. 1A) 1% Pt/SBA-15 (carbon-based), (FIG. 1B) 1% Pt/Si-KIT-6, (FIG. 1C) 1% Pt/Si-ZSM-5, (FIG. 1D) 1% Pt/Zr-KIT-6, (FIG. 1E) 1% Pt/Nb-KIT-6, and (FIG. 1F) 1% Pt/W-KIT-6 catalysts, 50 std cc/min hydrogen, 50 std cc/min nitrogen. In FIGS. 1A-1C, the lower bars correspond to catechol, the middle bars to phenol, and the upper bars (unlabeled) to all other products. In FIGS. 1D-1F, the lower bars correspond to 4-methylcatechol (4-Me-CAT), the middle bars to 4-methylphenol (4-Me-PHE), and the upper bars (unlabeled) to all other products.

FIGS. 2A-2B show temperature-programmed surface reaction (TPSR) profiles for (FIG. 2A) total pressure=0.1 MPa, and (FIG. 2B) total pressure=0.5 MPa, 50 std cc/min hydrogen, 50 std cc/min nitrogen, 0.025 ml/min guaiacol (liquid, room temperature), WHSV=33.3 h⁻¹.

FIG. 3 shows proposed overall reaction pathways of guaiacol hydrodeoxygenation over existing catalysts (top) and the present bifunctional catalysts (bottom).

FIGS. 4A-4C shows correlations between reaction rates of steps R4-R6 (FIG. Error! Reference source not found.) and number of sites for (FIG. 4A) Pt site, (FIG. 4B) Brønsted acid site, and (FIG. 4C) Lewis acid site, 50 std cc/min hydrogen, 50 std cc/min nitrogen, 0.025 ml/min guaiacol (liquid, room temperature) at 300° C., WHSV=33.3 h⁻¹.

FIG. 5 shows total pressure effect on guaiacol conversion and product distribution under standard operating conditions as follows: 50 mg 1%Pt/Nb-KIT-6 catalyst, 50 std cc/min hydrogen, 50 std cc/min nitrogen, 0.025 mL/min guaiacol (liquid, room temperature) at 300° C., WHSV=33.3 h⁻¹. The lower bars correspond to 4-Me-CAT, the middle bars to 4-Me-PHE, the next set of bars above to toluene, and the uppermost bars (unlabeled) to all other products.

FIG. 6 shows selectivity and conversion as a function of time, demonstrating the stability of the 1% Pt/Nb-KIT-6 catalysts. The conditions were as follows: 50 mg catalyst, 50 std cc/min hydrogen, 50 std cc/min nitrogen, 0.025 mL/min guaiacol (liquid, room temperature) at 300° C.

FIG. 7 shows proposed reaction schemes for guaiacol hydrodeoxygenation over the present bifunctional catalysts.

### DETAILED DESCRIPTION

Provided are methods for gas-phase deoxygenation of a bio-oil. In embodiments, a method for gas-phase deoxygenation of a bio-oil comprises exposing a bio-oil vapor, the bio-oil vapor comprising hydrocarbon compounds having oxygenated aromatic groups, to hydrogen gas in the presence of a transition metal-incorporated mesoporous silicate catalyst under conditions to induce deoxygenation of the oxygenated aromatic groups to provide a deoxygenated aromatic species. At least some embodiments of the present methods exhibit the advantage of both high bio-oil conversion and high selectivity of deoxygenated aromatic species. Moreover, these advantages are realized using relatively mild conditions (low temperature and hydrogen gas pressure).

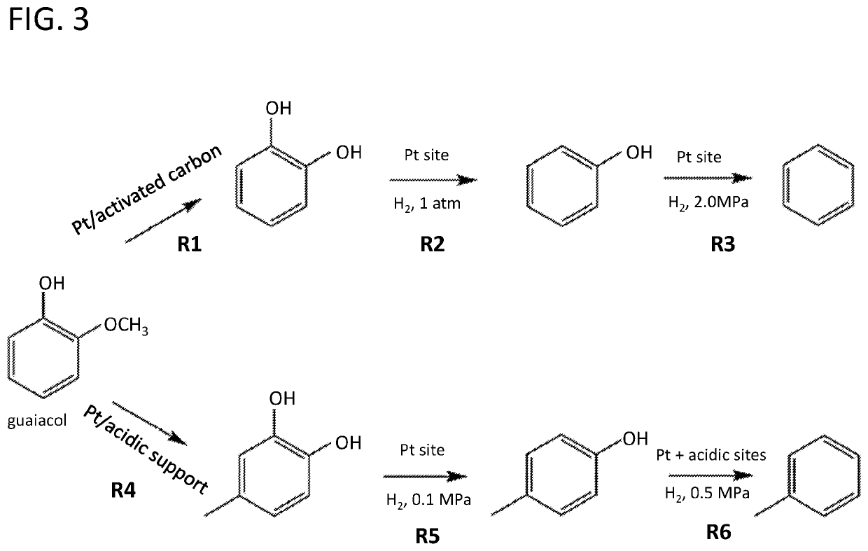

Bio-oil is a liquid fuel produced from the pyrolysis of lignocellulosic biomass. In such a process, the lignocellulosic biomass is thermally decomposed in the presence of an inert or reducing gas to various hydrocarbon compounds. Many of the hydrocarbon compounds comprise oxygenated aromatic groups derived from those present in the starting lignocellulosic biomass. Phenolic compounds such as phenol, guaiacol, and anisole are considered to be representative of the hydrocarbon compounds in bio-oil, and are often used as model compounds to assess the conversion and selectivity of bio-oil upgrading methods. The phenolic compound, guaiacol, is of particular interest as it comprises both hydroxyl (—OH) and methoxy (—OCH3) groups on the benzene ring. (See FIG. 3.)

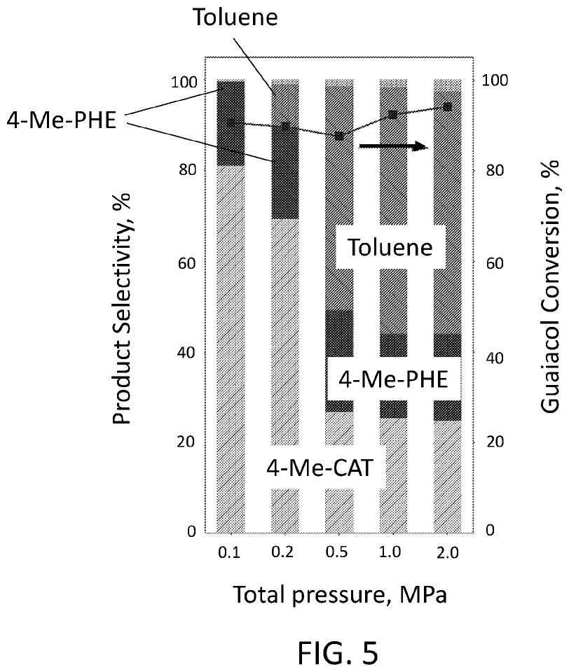

The present methods make use of a bio-oil vapor and are carried out in the gas-phase. This is by contrast to existing methods which are carried out in the liquid phase. That is, those existing methods involve pressures and/or temperatures such that the bio-oil remains in its liquid form and/or the deoxygenation reactions occur in the liquid-phase. (See, e.g., Lee, E. H. et al., Journal of lndustrial and Engineering Chemistry 37 (2016): 18-21.) The type of bio-oil used in the present methods is not particularly limited. The vaporized bio-oil is exposed to hydrogen gas. The pressure of the hydrogen gas may be adjusted to provide a desired conversion of bio-oil (e.g., maximum) and/or desired selectivity of a particular deoxygenated aromatic species (e.g., toluene, benzene). (See FIG. 5.) However, the H₂ pressure is sufficiently low so as not to liquify the bio-oil vapor/H₂ gas. In embodiments, the H₂ pressure is no more than 1.0 MPa, no more than 0.75 MPa, or no more than 0.5 MPa. This includes a H₂ pressure in a range of from 0.05 MPa to 1.0 MPa, from 0.25 MPa to 1.0 MPa, and from 0.1 MPa to 0.75 MPa. These H₂ pressures may be partial pressures if the hydrogen gas is combined with an inert carrier gas such as N₂. By way of illustration, in FIG. 5, the Example, below, the H₂ partial pressure is 50% of the total pressure indicated in the figure. Thus, total pressures of from 0.1 MPa to 2.0 MPa were used, with H₂ partial pressures of from 0.05 to 1.0 MPa.

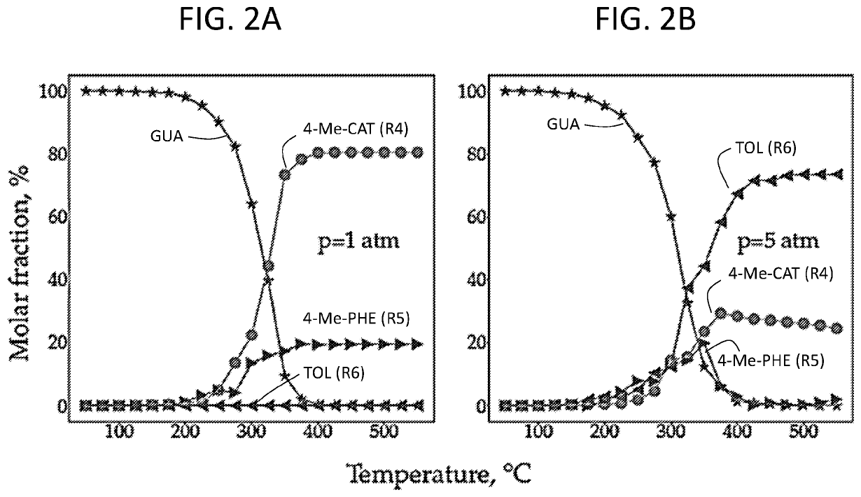

Besides H₂ pressure, the conditions under which the deoxygenation reactions are carried out refer to the temperature. The temperature may be adjusted to achieve a desired conversion and/or desired selectivity as described above, provided the temperature does not result in liquification. (See FIGS. 2A-2B.) in embodiments, the temperature is in a range of from 200° C. to 500° C. from 250° C. to 450° C., or from 250° C. to 400° C.

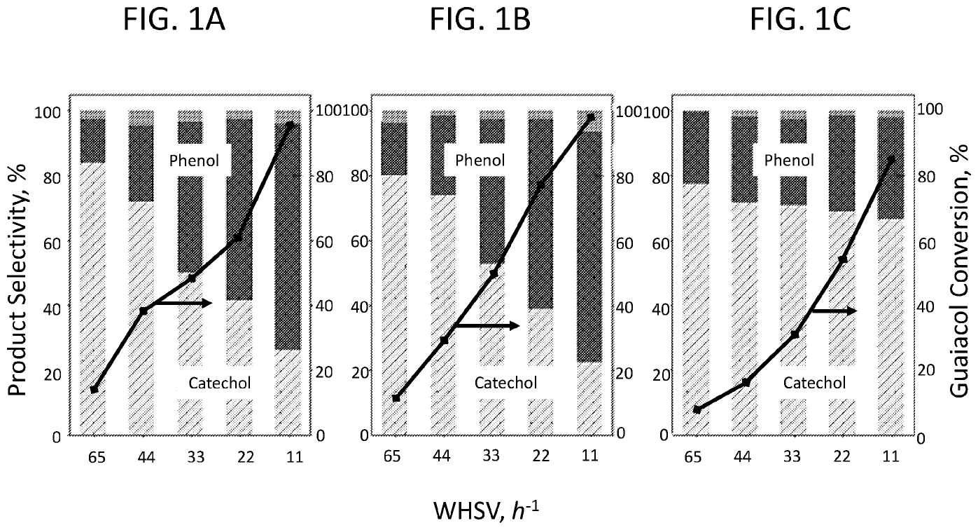

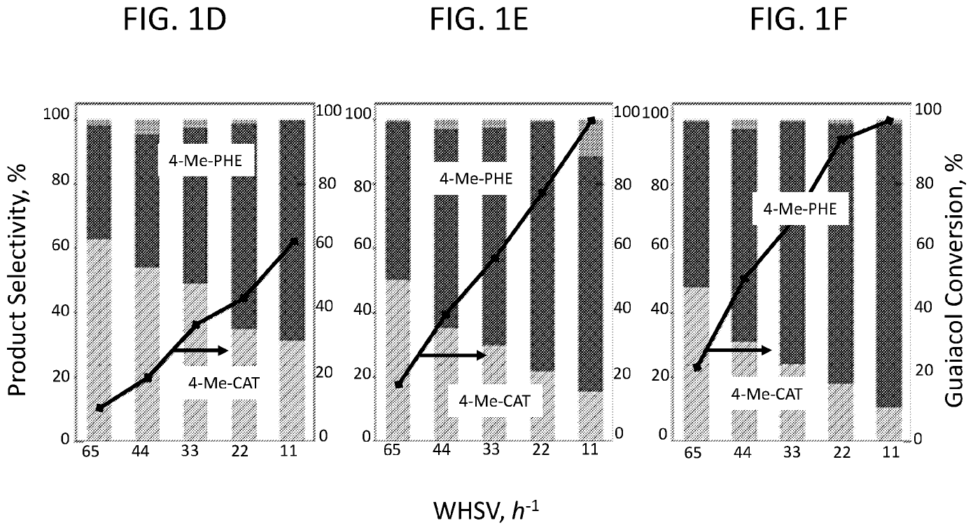

The present methods may be carried out using a variety of reactor systems. However, in embodiments, a flow reactor system (by contrast to a batch reactor system) is used. In such a flow reactor system, the bio-oil vapor and the H₂ gas are provided as a flowing stream to/over/through the transition metal-incorporated mesoporous silicate catalyst. A flow reactor system such as the fixed bed reactor system described in the Example below may be used. In such reactor systems, the conditions under which the deoxygenation reactions are carried out further refer to a weight hourly space velocity (WHSV). This parameter refers to the bio-oil vapor (gh⁻¹) per gram of catalyst (g). The WHSV may be adjusted to achieve a desired conversion and/or selectivity as described above. (See FIG. 1D-IF.) In embodiments, the WHSV is in a range of from 10 h⁻¹ to 40 h⁻¹, from 20 h⁻¹ to 40 h⁻¹, or from 30 h⁻¹ to 40 h⁻¹.

The catalyst used in the present methods a solid mesoporous silicate comprising transition metal atoms (M) incorporated therein and platinum (Pt) deposited thereon. The transition metal atoms incorporated into the mesoporous silicate may be selected from Nb, W, Zr, and combinations thereof. The terms “transition metal-incorporated” and the like refer to the direct incorporation of the transition metal atoms into the silicate lattice structure such that the transition metal atoms replace silicon atoms. This is by contrast to materials made using grafting and impregnation approaches which create active species without direct incorporation of the transition metal atoms into the silicate lattice structure. Evidence of transition metal-incorporation may be derived from small-angle X-ray scattering (SAXS) pattern analysis (to assess modification of the unit-cell parameters of the silicate lattice structure), elemental analyses (to assess amount of transition metal incorporation) as well as ultraviolet-visible spectroscopy (to determine existence of transition metal-oxygen bonding and bond structure).

Different types of mesoporous silicates may be used, each which may be characterized by its lattice structure and symmetry (e.g., as determined from SAXS). In embodiments, the catalyst is characterized by a cubic structure with Ia3d symmetry (known as “KIT-6”). In embodiments, the catalyst is characterized by a cubic structure with Fm3m symmetry (known as “KIT-5”). Other mesoporous silicates may be used including SBA-15.

By contrast to the incorporated transition metal atoms, the platinum of the catalyst is deposited on external surfaces of the transition-metal incorporated mesoporous silicate. This includes the platinum partially or fully filling pores defined by these external surfaces. The catalyst may be characterized by the loading of the platinum therein. In embodiments, the platinum loading is from 0.1% by weight to 5% by weight, from 0.1% by weight to 3% by weight, from 0.1% by weight to 2% by weight, or from 0.5% by weight to 1.5% by weight. The phrase “by weight” refers to the weight of the platinum as compared to the total weight of the catalyst as determined by inductively coupled plasma—atomic emission spectroscopy (ICP-AES) as described in the Example, below. The deposited platinum may be in the form of nanoparticles as evidenced by transmission electron microscope (TEM) images. These nanoparticles are generally quite small, e.g., having an average diameter in a range of from 0.1 nm to 20 nm, from 0.1 nm to 5 nm, or from 0.5 nm to 5 nm. TEM images may also be used to confirm uniform distribution of the platinum as well as the absence of the aggregation of platinum nanoparticles.

The catalyst may be further characterized by the ratio of Si to transition metal (M) atoms, i.e., Si/M. In embodiments, the Si/M ratio is in the range of from 5 to 100, including 5 to 50, and 10 to 40. Related to the Si/M ratio, the catalyst may be further characterized by its total acidity. Total acidity may be determined using temperature-programmed desorption of ammonia (NH₃-TPD) as described in the Example, below. (See Table 2.) In embodiments, the total acidity of the catalyst is in the range of from 0.10*10⁻⁶ kmol/kg_{cat} to 0.50*10⁻⁶ kmol/kg_{cat}, from 0.20*10⁻⁶ kmol/kg_{cat} to 0.40*10⁻⁶ kmol/kg_{cat}, or from 0.25*10⁻⁶ kmol/kg_{cat} to 0.35*10⁻⁶ kmol/kg_{cat} NH₃/g.

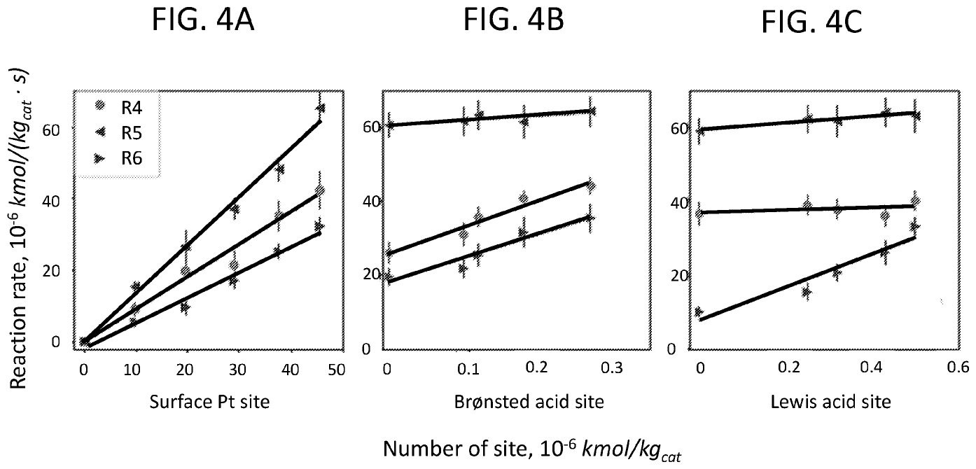

The catalyst may be characterized by the type of its acid sites. (See Table 2.) Acid sites may be Lewis acidic (i.e., exhibiting the ability to accept an electron pair) or Brønsted acidic (i.e., exhibiting the ability to provide a proton). In embodiments, the catalyst comprises both Lewis acid sites and Brønsted acid sites. As discussed in the Example, below, it has been found that both types of acid sites facilitate the deoxygenation of bio-oil to deoxygenated aromatic species, but high conversion/selectivity requires Brønsted acid sites. (See FIGS. 3 and 4A-4C). Acid type may be determined using pyridine-titrated Fourier-transform infrared spectroscopy (FTIR) as described in the Example, below.

The catalyst may be further characterized by properties such as surface area, pore diameter, and pore volume. (See Table 3.) The Brunauer—Emmett—Teller (BET) technique may be used to determine these properties as described in the Example, below. In embodiments, the surface area is in a range from 700 m²/g to 800 m²/g. In embodiments, the pore diameter is in the range of from 2 nm to about 20 nm, from 5 nm to about 15 nm, or from 6 nm to about 12 nm. These pore diameters distinguish the present catalysts from microporous catalysts, the latter which have smaller pore sizes. In embodiments, the pore volume is in a range of from 0.70 cm³/g to 1.00 cm³/g.

Using the guidance provided above, the particular catalyst, its characteristics and properties may be selected to provide a desired conversion of bio-oil and/or selectivity of a particular deoxygenated aromatic species. In embodiments, however, the catalyst is Pt/Nb-KIT-6. As described in the Example, below, this catalyst has been found to be unexpectedly superior as compared to certain comparative catalysts in being able to fully deoxygenate hydrocarbon compounds comprising oxygenated aromatic groups. Using guaiacol as a model such hydrocarbon compound, Pt/Nb-KIT-6 is able to convert as much as about 90% guaiacol to the fully deoxygenated aromatic species, toluene, with a selectivity of about 60%. Remarkably, this performance is achieved under mild conditions, i.e., 300° C., less than 1.0 MPa H₂ (2.0 MPa total pressure), and a WHSV of 33.3 h⁻¹. By contrast, under the same conditions, a comparative Pt/AC catalyst produced phenol (partially deoxygenated aromatic species) with a selectivity of less than 20% and no fully deoxygenated aromatic species were produced. The ability to fully deoxygenate guaiacol under mild conditions at such high selectivities, while also preserving aromaticity is unexpected in view of the otherwise close structural similarity of the Pt/AC catalyst.

Methods for making the catalysts may be adapted from those described in U.S. Pat. No. 9,994,601 and International Pat. Pub. No. WO 2019/147770, each of which is incorporated by reference in its entirety. The deposition of platinum is further described in the Example, below.

The hydrocarbons comprising oxygenated aromatic groups to be deoxygenated in the present methods are not particularly limited and depend upon the particular bio-oil being used. Thus, the particular deoxygenated species produced from the deoxygenation reactions depend upon the bio-oil. However, these deoxygenated species comprise deoxygenated aromatic species (as distinguished from species which may be deoxygenated, but are non-aromatic). The deoxygenated aromatic species may comprise partially deoxygenated aromatic species, fully deoxygenated aromatic species, or a combination thereof. By “partially deoxygenated,” it is meant that some oxygen has been removed from certain hydrocarbons in the bio-oil, but not all; thus, the partially deoxygenated aromatic species have one or more oxygen atoms therein. By “fully deoxygenated,” it is meant that all the oxygen has been removed from certain hydrocarbons in the bio-oil; thus, the fully deoxygenated aromatic species have no oxygen atoms therein. The phrase “deoxygenated aromatic species” encompasses both partially and fully deoxygenated aromatic species. In embodiments, no deoxygenated, non-aromatic species are produced. This means that the amount of such species is 0% by weight or less than 2% by weight, as compared to the total weight of deoxygenated species.

The present methods are characterized by the ability to achieve high conversion of the bio-oil hydrocarbons as well as high selectivities of deoxygenated aromatic species, including fully deoxygenated aromatic species. Although some existing methods achieve high conversion, such methods may suffer from low selectivity of deoxygenated aromatic species (e.g., tendency to hydrogenate aromatic rings, resulting in deoxygenated, but non-aromatic species) and/or low selectivity of fully deoxygenated aromatic species (e.g., tendency to form only partially deoxygenated aromatic species). This is true of existing methods such as Lee, E. H. et al., Journal of Industrial and Engineering Chemistry 37 (2016): 18-21 and U.S. Pat. No. 9,783,474.

In embodiments, the present methods achieve a bio-oil conversion of at least 85%, at least 90%, or at least 95%. In embodiments, the present methods achieve a selectivity of deoxygenated aromatic species of at least 70%, at least 75%, or at least 80%. In embodiments, the present methods achieve a selectivity of fully deoxygenated aromatic species of at least 45%, at least 50%, at least 55%, or at least 60%. The conversion/selectivity values for the present methods may be determined by carrying out the present methods with a model compound, e.g., guaiacol, in place of the bio-oil. The conversion/selectivity values in this paragraph may refer to those obtained using such a model compound. In such cases, guaiacol conversion may be determined as (mol of converted guaiacol)/(mol of feed guaiacol)*100 and product selectivity may be determined as (mol of product)/(mol of converted guaiacol)*100. The conversion/selectivity values in this paragraph may also refer to a specific set of conditions, e.g., 50 mg catalyst, 50 standard cubic centimeters per minute H₂, 50 standard cubic centimeters per minute N₂, 0.025 mL/min guaiacol, 300° C., WHSV 33.3 h⁻¹.

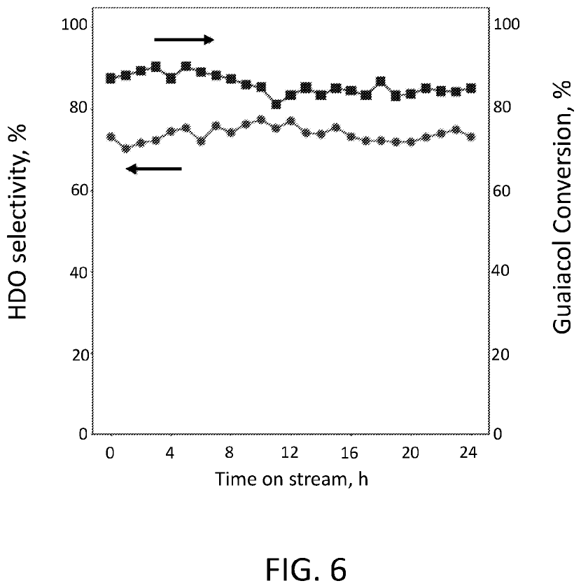

At least some of the catalysts used in the present methods advantageously exhibit high stability. This may be confirmed by monitoring guaiacol conversion/ selectivity of deoxygenated aromatic species over time. In embodiments, the catalysts used under a certain set of conditions (50 mg catalyst, 50 standard cubic centimeters per minute H₂, 50 standard cubic centimeters per minute N₂, 0.025 mL/min guaiacol, 300° C., WHSV 33.3 h⁻¹) achieve a guaiacol conversion and a selectivity of deoxygenated aromatic species after 24 hours that is the same (i.e., within ±10%, ±5%, ±2%) as these values at an initial time point. (See FIG. 6.) Stability may also be confirmed by measuring coke formation on the catalysts after they are used under the conditions described in this paragraph for 24 hours. Coke formation may be measured as described in the Example, below. In embodiments, the coke formation measured in this way is no more than 60 mg/g_{cat}, no more than 50 mg/g_{cat}, or in a range of from 40 mg/g_{cat} to 60 mg/g_{cat}.

The present disclosure also encompasses the catalysts described herein, including Pt/W-KIT-6 and Pt/Nb-KIT-6.

### EXAMPLE

### Introduction

Owing to high oxygen content, bio-oils from fast pyrolysis of lignin are upgraded to meet the fuel specification standard. However, catalytic hydrodeoxygenation (HDO) of bio-oils faces several challenges such as low hydrocarbon yield, the requirement of high operating pressure for full deoxygenation, and catalyst deactivation caused by coking. In the present work, various Pt-based bifunctional catalysts consisting of bimetallic components (Pt-Nb, Pt-W or Pt-Zr) supported over mesoporous and acidic KIT-6 were prepared, characterized, and tested for gas-phase HDO of guaiacol, a widely used model compound of bio-oil. It was found that full HDO of guaiacol with˜90% conversion and˜80% hydrocarbon selectivity occurred over 1% Pt/Nb-KIT-6 under relatively mild operating conditions, 300° C., 0.5 MPa H₂ and WHSV (weight hourly space velocity) of 33 h⁻¹. No significant deactivation was observed for a 24-h continuous run. Mechanistic investigations indicate that acidic supports promote transalkylation reactions, which benefit the enhancement of aromatic hydrocarbon yields. A reaction mechanism including the active catalytic sites associated with the various reaction steps were identified by correlating with experimentally measured reaction rates. The results show that the catalysts may be used for bio-oil upgrading under mild conditions.

### Materials and Catalyst Synthesis

Hexachloroplatinic acid hexahydrate (H₂PtCl₆·H₂O, >99.9% metal basis from Sigma Aldrich) was used as Pt precursor. The 60-80 mesh activated carbon (AC) supports were from Norit Americas Inc. Guaiacol (>98.0%) (GUA) and all other calibration compounds, including catechol (CAT), phenol (PHE), cyclopentanone, 4-methylcatechol (4-Me-CAT), 4-methylphenol (p-cresol) (4-Me-PHE), and toluene, were purchased from Sigma-Aldrich. Ultra high purity grade gases such as O₂ (99.999%), Ar (99.99%), N₂ (99.999%), He (99.98%), and H₂ (99.999%), were purchased from Indiana Oxygen. The 0.5% Pt/Al₂O₃ (metal dispersion=31±0.5%) standard, from Micromeritics, was used for calibration in H₂-O₂ titration experiments. The Pt-based catalysts were prepared by an incipient wetness impregnation procedure.

### Catalyst Characterization

Included below are the detailed operating conditions and equipment information for catalyst characterization techniques, including BET (Brunauer-Emmett-Teller), TPSR (temperature-programmed surface reaction), H₂-O₂ titration, ICP-AES (inductively coupled plasma—atomic emission spectroscopy), TEM (transmission electron microscopy), TPO (temperature-programmed oxidation), pyridine-titrated FTIR (Fourier-transform infrared spectroscopy), and NH₃-TPD (temperature-programmed desorption).

BET measurements were conducted by N₂ adsorption and desorption at 77 K via a Micromeritics ASAP 2020 apparatus, giving physisorption properties of catalysts, including surface area, pore size, and pore volume. Before measurements, degassing was carried out at 300° C. for 8 hrs. For Pt-loaded catalysts supported on various mesoporous materials, H₂-O₂ titration was carried out at room temperature. The TEM scans were obtained at 200 kV with LaB₆ source (FEI-Tecnai). The TEM samples were prepared by suspending fine catalyst particles in ethanol, followed by dispersing them on 200 copper mesh grids with lacey carbon film coating, and then drying in air at room temperature. Elemental analysis of catalysts was carried out by the ICP-AES method (SPECTRO Instrument). For TPSR experiments, the catalyst was reduced using 5 vol% H₂ at 400° C. for 2 hrs before the TPSR measurement. Upon cooling to room temperature, a 1% reactant in 10% H₂ gas mixture flow was fed at 20 std cc/min, and the temperature was increased from room temperature to 550° C. at heating rate 5° C./min. A TCD was used to detect products. In the TPO process, 5% O₂ in N₂ gas mixture was used as the oxidizing gas. The used catalysts were packed in the reactor for TPO measurements without any pretreatment, while the heating rate was 5° C./min as well.

FTIR spectra of adsorbed pyridine were obtained on a Tensor-27 instrument with Pike diffuse reflectance accessory. Prior to measurements, samples were heated to 450° C. for 2 h in a flow of He gas, followed by cooling down to room temperature. Then samples were wetted with pyridine followed by evacuation of physisorbed pyridine at 120° C., and the FTIR spectra were measured at three different temperatures such as 120, 250, and 400° C., giving essentially the same acid site numbers. Since the guaiacol HDO reaction was typically tested at 300° C., measurements of acid site numbers correspond to those at the working temperature. The results clearly indicate the presence of both the Lewis acid sites (1455 cm⁻¹) and Bronsted acid sites (1550 cm⁻¹). NH₃-TPD experiments were performed using a Micromeritics Autochem II 2920 Chemisorption analyzer equipped with an Agilent 5975C mass selective detector (MSD) to identify the gaseous species evolved from the catalyst samples. The samples were placed between two quartz wool plugs inside a U-shaped quartz cell held within a clamshell furnace, with 50 cm³/min flowing UHP helium at ambient temperature for 1 h, and heated to 873 K (0.167 K/s).

### Catalytic Performance Test and Product Analysis

The catalytic performance tests were conducted in a fixed-bed reactor. Prior to the test, activation of packed catalyst was performed at 723 K for 4 hours under a H₂-N₂ mixture flow (H₂:N₂=1:2, 100 std cc/min). The reactor was then purged by nitrogen with a flow of 50 std cc/min for 15 min. Differential/integral reactions were performed by varying packed amount of catalysts, leading to different conversion values of reactants, where differential reactions refer to conversion values smaller than 10%, while integral reactions correspond to greater than 30% conversion. The following standard operating conditions were used: 50 mg catalyst, 50 std cc/min hydrogen, 50 std cc/min nitrogen, 0.025 ml/min guaiacol (liquid, room temperature) at 300° C. All experiments have carbon mass balances, including both liquid and gas phases, of 94±2%. Unless stated otherwise, all data sets were taken at 10 min time on stream (TOS) as initial reaction rates. GC (Agilent GC6890) with flame ionization detector (FID), equipped with a DB-1701 column (30 m×0.25 mm) was used for quantitative analysis of the liquid products. The gaseous effluent was analyzed using a Micro GC (Agilent 3000A Micro GC) equipped with two columns (Column A, MolSieve 5 A, 10 m×0.32 mm; Column B: Plot U, 8 m×0.32 mm) and two thermal conductivity detectors (TCD). For the reaction experiments, good repeatability generally within less than 2% deviation was achieved for all quantitative analysis. All data for catalytic performance were taken at 10 min time on stream (TOS).

### Results and Discussion

### Initial Performance of Various Mesoporous Bifunctional Catalysts for Guaiacol HDO

Various mesoporous bifunctional catalysts were tested: 1 wt % Pt loaded over carbon-SBA-15, Si-KIT-6, Nb-KIT-6 (Si/Nb=20), W-KIT-6 (Si/W=20), and Zr-KIT-6 (Si/Zr=20). As comparison, 1 wt % Pt over microporous Si-ZSM-5 (no Al in the MFI framework) was also used. Under standard operating conditions (300° C., atmosphere pressure, H₂: guaiacol molar ratio=10), initial catalytic performance [based on data taken at TOS (time on stream)=10 min] of these catalysts is shown in FIGS. 1A-1F. As inferred from FIGS. 1A-1B, Pt/SBA-15 and Pt/Si-KIT-6 catalysts essentially exhibited the same distribution of product species, including catechol as un-deoxygenated species and phenol as partially deoxygenated species. Note that no fully deoxygenated species (such as benzene or toluene) was observed. The guaiacol conversion as well as partial deoxygenation performance (i.e. phenol selectivity) improved with decreased WHSV for both 1% Pt/SBA-15 (FIG. 1A) and 1% Pt/Si-KIT-6 (FIG. 1B). For Pt/Si-ZSM-5 (FIG. 1C), although guaiacol conversion values increased up to 90%, the selectivity to phenol (˜20-30%) and catechol (˜70-80%) changed less compared to the other catalysts. The carbon-SBA-15 and Si-KIT-6 are mesoporous materials, while Si-ZSM-5 is microporous. Thus, the mesoporous supports perform better, likely owing to easier accessibility of substrates as compared to microporous materials. Interestingly, as seen in FIGS. 1D-1F, 4-methylcatcheol (4-Me-CAT) and 4-methylphenol (4-Me-PHE) were observed as major products over Pt-loaded acidic mesoporous supports (1% Pt/Zr-KIT-6, 1% Pt/Nb-KIT-6, and 1% Pt/W-KIT-6). On these three catalysts (FIGS. 1D-1F), the selectivity towards deoxygenated species was generally favored at lower space velocities. The lower guaiacol conversion over 1% Pt/Zr-KIT-6 catalyst (FIG. 1D) is attributed to a lack of Bronsted acid sites, which is addressed in a later section. Both 4-Me-CAT and 4-Me-PHE are derived from transalkylation of guaiacol, which was likely promoted by acidic sites on these bifunctional catalysts. In fact, transalkylation of guaiacol increased the yield of aromatics, which is desirable for enhancing fuel quality. As compared with non-acidic supports, the Pt loaded acidic mesoporous materials generate entirely different species. The reaction network and active sites associated with these steps are discussed in a later section.

### Reaction Pathway and Effects of Operating Conditions

Although Pt-based acidic mesoporous catalysts exhibit promising HDO performance of guaiacol (FIGS. 1A-1F), the products are still partially deoxygenated. In existing HDO methods, full HDO of guaiacol requires high H₂ pressure. As shown in FIG. 5, at 0.1 MPa total pressure (0.05 MPa hydrogen), only 4-Me-CAT (4-methylcatechol) and 4-Me-PHE (4-methylphenol) were generated using 1% Pt/Nb-KIT-6. However, when the H₂ partial pressure was increased by a small amount (only 0.1 MPa), some toluene (selectivity˜10%) was observed. Surprisingly, at still higher H₂ pressures (0.5-1.0 MPa), the major product was toluene (selectivity˜55%), followed by the undeoxygenated species (4-Me-CAT) with a selectivity of ˜25% and the partially deoxygenated species (4-Me-PHE) with a selectivity of ˜20%. Over the entire pressure range (0.1-1 MPa), the guaiacol conversion was essentially constant. These observations indicate that the H₂ partial pressure plays an important role in guaiacol HDO, specifically by converting 4-methylcatechol to 4-methylphenol, followed by converting 4-methylphenol to toluene. In contrast, only a trace amount of fully deoxygenated species (benzene) was produced when the reaction was operated with 0.1-1.5 MPa H₂ over the non-acidic, mesoporous AC support. Much higher H₂ pressures (2.0-2.5 MPa) were required to achieve benzene selectivity of between 70-80%.

To further investigate the effects of operating temperature and pressure on guaiacol HDO performance, TPSRs (temperature-programmed surface reactions) at 0.1 MPa and 0.5 MPa total pressure (0.05 MPa and 0.25 MPa hydrogen gas partial pressure, respectively) were carried out in the temperature range of 50-550° C. At both operating pressures, guaiacol was activated at about 250° C., reaching full conversion at about 400° C. (FIGS. Error! Reference source not found.A-2B). When the H₂ partial pressure was 0.05 MPa (total pressure 0.1 MPa), only 4-methylphenol and 4-methylcatechol (selectivity values being 20% and 80% respectively) were generated over the entire temperature range. Only traces of toluene (<0.1%) were observed as byproduct. This implies that temperature does not influence hydrodeoxygenation activity at the lower H₂ partial pressure. However, when the total pressure was increased to 0.5 MPa (0.25 MPa H₂ partial pressure), a surprising and significant amount of toluene was produced even at a relatively low temperature (300° C.). Remarkably, the selectivity to 4-Me-PHE dropped beyond 350° C., indicating that at relatively high temperatures, 4-Me-PHE was converted to toluene. Based on these experimental observations, a reaction pathway is proposed in FIG. Error! Reference source not found., in which steps R1-R3 demonstrate guaiacol HDO over non-acidic supports, while steps R4-R6 refer to guaiacol HDO over acidic supports.

As shown in FIG. 6, no significant catalyst deactivation, in terms of both guaiacol conversion and selectivity to deoxygenated species, was observed during the 24-hour run.

Further, TPO (Temperature-programmed oxidation) studies reveal that only a small amount of coke (˜52 mg/g_{cat}) formed during the 24-hour run, indicating the excellent coke-resistant property of the 1% Pt/Nb-KIT-6 catalyst. Note that for a non-coke-resistant catalyst, the amount of coke reaches ˜500 mg/g_{cat} within several hours. (Xiao Y, et al., ACS Catalysis. 2018;8(4):2735-2740.)

### Insights into Active Sites

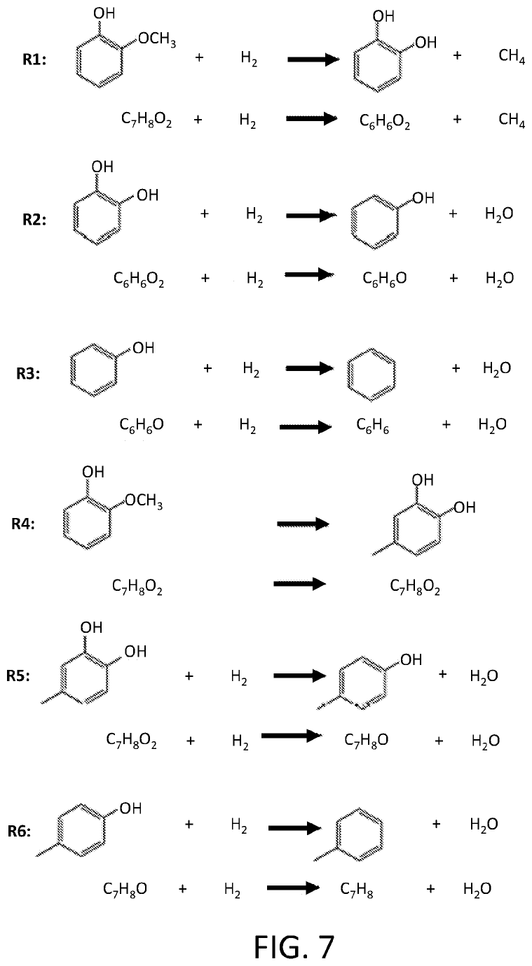

The active sites on the bifunctional catalysts were systematically probed for each of the reaction steps (R4-R6) shown in FIG. Error! Reference source not found. The proposed reaction schemes associated with steps R1-R6 are shown in FIG. 7. Three possible active sites were considered as follows: surface Pt sites measured by H₂-O₂ titration, Brønsted acid and Lewis acid sites measured by pyridine-IR at 1550 and 1455 cm⁻¹, respectively. As shown in Table 1, the calcination temperature affects the acidity of supports. Using the same acidic support (W-KIT-6), various amounts (0-1%) of Pt were loaded (C06, C11-C15 in Table 2) at essentially constant metal dispersion (89-95%) and nearly identical Pt particle size (˜1 nm). TEM micrographs confirm that the particle size of Pt is relatively uniform at various metal loadings (0.2-1%). The various catalysts exhibit similar surface areas (700-800 m²/g), pore size (8.5-10 nm) and pore volume (0.7-1.0 cm³/g) at the targeted Pt loading (Table 3). Further, both the number of Brønsted acid (0.26-0.27×10⁻⁶ kmol/kg_{cat}) and Lewis acid (0.05-0.06×10⁻⁶ kmol/kg_{cat}) sites are nearly identical on these catalysts (Table 2). Thus, only one parameter, the number of surface Pt sites, was varied. Similarly, only the number of Lewis acid sites was varied in Pt/Zr-KIT-6 (C16-C19 in Table 2) and Pt/Si-KIT-6 (C20 in Table 2), and the number of Brønsted acid sites was varied in the Pt/W-KIT-6 and Pt/Nb-KIT-6 samples (C15, C21-C24 in Table 2).

To individually investigate the reaction steps R4-R6 (FIG. Error! Reference source not found.), the intrinsic rate of each step on the various bifunctional catalysts was measured under differential operating conditions (reactant conversion <10%) by separately co-feeding guaiacol (R4), 4-methylcatechol (R5), or 4-methylphenol (R6) with hydrogen. As shown in FIG. 4A, the reaction rates of steps R4-R6 correlate linearly with the number of surface Pt sites. Remarkably, all these three reaction rates were negligible without surface Pt, even though acidic sites (both Brønsted and Lewis) were present. Further, the rate of step R5 was essentially independent of the number of either Brønsted or Lewis acid sites (FIGS. 4B-4C). These indicate that the active site for step R5, a hydrodeoxygenation reaction, was solely the surface Pt site, but not any acidic sites. For step R4, while the reaction rate correlated well with the number of Brønsted acid sites, it was relatively independent of the Lewis acid sites. Note from FIG. 4B, that when the number of Brønsted acid sites was close to zero, significant reaction rates were still observed. This implies that in addition to Brønsted acid sites, the Pt sites also promoted step R4. Interestingly, all three curves for step R6 correlate linearly with Pt, Brønsted, and Lewis acid sites. In addition, as seen from FIGS. 4B-4C, both Brønsted and Lewis acid sites accelerated the rate when the required number of Pt sites were present.

TABLE 1
Properties of various unsupported catalyst samples (no Pt).

| | | | ×10⁻⁶ kmol/kg_{cat} | | |
|---|---|---|---|---|---|
| Catal.# | Sup. | calc. temp. ° C. | Brønsted | Lewis | Total acid |
| C01 | SBA-15 | 550 | 0 | 0 | 0 |
| C02 | Si-KIT-6 | 550 | 0 | 0 | 0 |
| C03 | Si-ZSM-5 | 550 | 0 | 0 | 0 |
| C04 | Zr-KIT-6 | 550 | 0 | 0.51 | 0.51 |
| C05 | Nb-KIT-6 | 550 | 0.18 | 0.06 | 0.24 |
| C06 | W-KIT-6 | 550 | 0.27 | 0.06 | 0.33 |
| C07 | Zr-KIT-6 | 650 | 0 | 0.32 | 0.32 |
| C08 | Nb-KIT-6 | 650 | 0.12 | 0.04 | 0.16 |
| C09 | W-KIT-6 | 650 | 0.10 | 0.05 | 0.15 |
| C10 | Zr-KIT-6 | 750 | 0 | 0.26 | 0.26 |

TABLE 2
Properties of various supported catalyst samples (including Pt). The column “Pt disp.”
refers to the platinum dispersion, the percent ratio of the total number of metal atoms
present on the surface and the total number of metal atoms including surface and bulk.

| | | Pt load. | Pt disp., | calc. | ×10⁻⁶ kmol/kg_{cat} | | | |
|---|---|---|---|---|---|---|---|---|
| Catal.# | Sup. | % | % | temp. ° C. | Pt | Brønsted | Lewis | Total acid |
| C06 | W-KIT-6 | 0 | — | 550 | 0 | 0.27 | 0.06 | 0.33 |
| C11 | W-KIT-6 | 0.2 | 95 | 550 | 9.7 | 0.27 | 0.06 | 0.33 |
| C12 | W-KIT-6 | 0.4 | 95 | 550 | 19.5 | 0.27 | 0.06 | 0.33 |
| C13 | W-KIT-6 | 0.6 | 94 | 550 | 28.9 | 0.26 | 0.06 | 0.32 |
| C14 | W-KIT-6 | 0.8 | 92 | 550 | 37.7 | 0.26 | 0.05 | 0.31 |
| C15 | W-KIT-6 | 1.0 | 89 | 550 | 45.6 | 0.26 | 0.06 | 0.32 |
| C16 | Zr-KIT-6 | 1.0 | 94 | 550 | 47.7 | 0 | 0.50 | 0.50 |
| C17 | Zr-KIT-6 | 1.0 | 93 | 600 | 47.2 | 0 | 0.43 | 0.43 |
| C18 | Zr-KIT-6 | 1.0 | 92 | 650 | 47.0 | 0 | 0.32 | 0.32 |
| C19 | Zr-KIT-6 | 1.0 | 92 | 750 | 46.9 | 0 | 0.25 | 0.25 |
| C20 | Si-KIT-6 | 1.0 | 94 | 550 | 47.8 | 0 | 0 | 0 |
| C15 | W-KIT-6 | 1.0 | 89 | 550 | 45.6 | 0.27 | 0.06 | 0.33 |
| C21 | Nb-KIT-6 | 1.0 | 93 | 550 | 47.6 | 0.18 | 0.06 | 0.24 |
| C22 | Nb-KIT-6 | 1.0 | 92 | 650 | 47.1 | 0.12 | 0.04 | 0.16 |
| C23 | W-KIT-6 | 1.0 | 92 | 650 | 47.1 | 0.10 | 0.05 | 0.15 |
| C24 | Zr-KIT-6 | 1.0 | 92 | 900 | 46.9 | 0 | 0.07 | 0.07 |

TABLE 3
ICP-AES elemental analysis and BET surface
area of various catalyst samples.

| | | | Surface | Pore | Pore volume, |
|---|---|---|---|---|---|
| Catal.# | Catal. sample ¹ | Pt % ² | area, m²/g | size, nm | cm³/g |
| C06 | unsupported | 0 | 742 | 9.1 | 0.82 |
| | W-KIT-6 | | | | |
| C11 | 0.2% Pt/W-KIT-6 | 0.19 | 713 | 8.4 | 0.75 |
| C15 | 1.0% Pt/W-KIT-6 | 0.98 | 724 | 8.5 | 0.77 |
| C16 | 1.0% Pt/Zr-KIT-6 | 0.97 | 795 | 9.4 | 0.95 |
| C19 | 1.0% Pt/Zr-KIT-6 | 0.96 | 774 | 9.2 | 0.93 |
| C23 | 1.0% Pt/W-KIT-6 | 0.98 | 705 | 8.8 | 0.72 |

¹ The Pt weight percentage is based on the amount used in catalyst preparation procedure;
² The Pt weight percentage refers to the actual value measured by ICP-AES analysis.

### Conclusion

In this Example, various Pt-based bifunctional catalysts supported over mesoporous metal-exchanged silicates (Zr-KIT-6, W-KIT-6 and Nb-KIT-6) with varying Pt, Lewis, and Brønsted acid sites were prepared, characterized, and tested for HDO of guaiacol, a widely used model compound of bio-oil. It was found that the acidic supports promoted transalkylation reactions, which enhanced aromatic hydrocarbon yields. Full HDO was observed on these catalysts at total pressures ranging from 2 MPa to 0.5 MPa. An HDO reaction pathway consisting of several reaction steps along with correlations between the rates of the individual reaction steps and either the metal and/or acid function were delineated. In particular, it was demonstrated that 1% Pt/Nb-KIT-6 provided enhanced and stable guaiacol HDO performance with ˜90% guaiacol conversion and ˜80% selectivity to deoxygenated species during the entirety of a 24-hour test. This Example demonstrates catalysts which may be used for commercial bio-oil upgrading under mild conditions and provides insights into active site requirements for various reactions involved in bio-oil upgrading.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the invention to enable one skilled in the art to utilize the invention in various embodiments and with various modifications as suited to the particular use contemplated. It is intended that the scope of the disclosure be defined by the claims appended hereto and their equivalents.

What is claimed is:

1. A method for gas-phase deoxygenation of a bio-oil, the method comprising: exposing a bio-oil vapor comprising hydrocarbon compounds having oxygenated aromatic groups, to hydrogen gas in the presence of catalyst under conditions to induce deoxygenation of the oxygenated aromatic groups to provide a deoxygenated aromatic species, wherein the catalyst is a transition metal-incorporated mesoporous silicate having platinum deposited thereon and the transition metal is selected from Nb, W, Zr, and combinations thereof.

2. The method of claim 1, wherein the hydrogen gas has a pressure of no more than 1.0 MPa.

3. The method of claim 2, wherein the hydrogen gas has a pressure of no more than 0.5 MPa.

4. The method of claim 1, wherein the hydrogen gas has a pressure of no more than 1.0 MPa and the step of exposing uses a temperature in a range of from 200° C. to 500° C. and a weight hourly space velocity in a range of from 10 h⁻¹ to 40 h⁻¹.

5. The method of claim 1, wherein the mesoporous silicate is KIT-6.

6. The method of claim 1, wherein the catalyst comprises both Lewis acid sites and Brønsted acid sites.

7. The method of claim 1, wherein the catalyst is Pt/Nb-KIT-6, Pt/W-KIT-6, or Pt/Zr-KIT.

8. The method of claim 1, wherein the catalyst is Pt/Nb-KIT-6 or Pt/W-KIT-6.

9. The method of claim 1, wherein the catalyst is Pt/Nb-KIT-6.

10. The method of claim 1, wherein at a hydrogen gas pressure of no more than 1.0 MPa, a temperature in a range of from 200° C. to 500° C., and a weight hourly space velocity in a range of from 10 h⁻¹ to 40 h⁻¹, the deoxygenated aromatic species comprise fully deoxygenated aromatic species.

11. The method of claim 1, wherein at a hydrogen gas pressure of no more than 1.0 MPa, a temperature in a range of from 200° C. to 500° C., and a weight hourly space velocity in a range of from 10 h⁻¹ to 40 h⁻¹, the method achieves a conversion of the bio-oil of at least 85% and a selectivity of the deoxygenated aromatic species of at least 70%.

12. The method of claim 11, wherein the deoxygenated aromatic species comprise fully deoxygenated aromatic species.

13. The method of claim 12, wherein the method achieves a selectivity of the fully deoxygenated aromatic species of at least 45%.

14. The method of claim 1, wherein at a hydrogen gas pressure of no more than 1.0 MPa, a temperature in a range of from 200° C. to 500° C., and a weight hourly space velocity in a range of from 10 h⁻¹ to 40 h⁻¹, the method does not produce deoxygenated non-aromatic species.

15. A catalyst for gas-phase deoxygenation of a bio-oil, wherein the catalyst is a transition metal-incorporated mesoporous silicate having platinum deposited thereon and the transition metal is selected from Nb, W, Zr, and combinations thereof.

16. The catalyst of claim 15, wherein the mesoporous silicate is KIT-6.

17. The catalyst of claim 15, wherein the catalyst comprises both Lewis acid sites and Brønsted acid sites.

18. The catalyst of claim 15, wherein the catalyst is Pt/Nb-KIT-6, Pt/W-KIT-6, or Pt/Zr-KIT.

19. The catalyst of claim 15, wherein the catalyst is Pt/Nb-KIT-6 or Pt/W-KIT-6.

20. The catalyst of claim 15, wherein the catalyst is Pt/Nb-KIT-6.
